(19) United States
(12) Patent Application Publication
(10) Pub. No.: US 2024/0269482 A1
(43) Pub. Date: Aug. 15, 2024

(54) MEDICAL DEVICES, SYSTEMS, AND RELATED METHODS

(71) Applicant: Boston Scientific Scimed, Inc., Maple Grove, MN (US)

(72) Inventors: Evan BENNETT, Minneapolis, MN (US); Aldo MORA ALVARADO, Alajuela (CR); Ricardo Andres ESQUIVEL DAVANZO, Cartago (CR); Timothy Lawrence RUBESCH, Blaine, MN (US); Peter FADLOVICH, Minnetonka, MN (US); Lawrence ABOYER, Indianapolis, IN (US); Paige O'REILLY, Mendota Heights, MN (US)

(73) Assignee: Boston Scientific Scimed, Inc., Maple Grove, MN (US)

(21) Appl. No.: 18/437,356

(22) Filed: Feb. 9, 2024

Related U.S. Application Data
(60) Provisional application No. 63/484,237, filed on Feb. 10, 2023.

(51) Int. Cl. A61N 5/10; A61M 25/10

(52) U.S. Cl. CPC A61N 5/10; A61M 25/1011; A61M 25/10182; A61M 2025/1061; A61M 2025/1079; A61M 2210/1053; A61M 2210/1096; A61M 2210/167; A61N 2005/1094; A61N 2005/1096

(57) ABSTRACT

A medical device for use during radiation therapy may include a longitudinal body configured to be positioned within a urethra. The longitudinal body may include a lumen, a distal end, and a proximal end. A first balloon may be coupled to a distal portion of the longitudinal body and fluidly connected to a first port at the proximal end of the longitudinal body. A second balloon may be positioned between the first balloon and the proximal end of the longitudinal body and fluidly connected to a second port at the proximal end of the longitudinal body.

### CROSS-REFERENCE TO RELATED APPLICATION(S)

This patent application claims the benefit of priority under 35 U.S.C. § 119 to U.S. Provisional Patent Application No. 63/484,237, filed Feb. 10, 2023, which is herein incorporated by reference in its entirety.

### TECHNICAL FIELD

Various aspects of the present disclosure relate generally to systems, devices, and methods for improving radiation therapy. More specifically, aspects of the disclosure pertain to systems, devices, and/or methods for penile bulb repositioning and/or radiation blocking, via medical devices, such as catheter assemblies.

### BACKGROUND

Radiation therapy uses high-energy rays or particles to kill cancer cells. Radiation therapy may be used to treat, for example, prostate cancer. Intensity-modulated radiation therapy (IMRT) includes using, for example, ions and intensity-modulated electromagnetic radiation therapy using, for example, x-rays for therapy applied to a subject. IMRT is a common type of external beam radiation therapy for prostate cancer. It uses a computer-driven machine that moves around the patient as it delivers radiation. Along with shaping the beams and aiming them at the prostate from several angles, the intensity (strength) of the beams can be adjusted to limit the doses of radiation reaching nearby normal tissues. This lets doctors deliver an even higher radiation dose to the cancer, while limiting radiation delivered to normal tissues. However, approximately 25-50% of men who undergo IMRT as a treatment for prostate cancer experience erectile dysfunction in years following treatment. Radiation dose to the penile bulb is associated with potency. For example, in a recent study of 276 patients, a mean radiation dosage of 20 Gy or higher to the penile bulb was predictive of erectile dysfunction.

Therefore, a need exists for systems, devices, and/or methods for repositioning the penile bulb to avoid radiation and/or blocking the penile bulb from radiation during radiation treatment for prostate cancer.

### SUMMARY

Aspects of this disclosure include medical systems and devices, comprising a catheter and methods of use thereof, e.g., methods of delivering a catheter to a target site of a patient and/or methods of operating a catheter, for example, to move a penile bulb of a patient and/or to block a penile bulb of a patient from receiving radiation. Each of the aspects disclosed herein may include one or more of the features described in connection with any of the other disclosed aspects.

According to at least one aspect, a medical device for use during radiation therapy may comprise a longitudinal body configured to be positioned within a urethra. The longitudinal body may include a lumen, a distal end, and a proximal end. A first balloon may be coupled to a distal portion of the longitudinal body and fluidly connected to a first port at the proximal end of the longitudinal body. A second balloon may be positioned between the first balloon and the proximal end of the longitudinal body and fluidly connected to a second port at the proximal end of the longitudinal body.

Any of the aspects disclosed herein may have any of the following features, alone or in any combination. The medical device may be a urinary catheter. The medical device may further comprise at least one marker proximate to the second balloon. The at least one marker may be a first marker, and the medical device may further comprise a second marker. The first marker may be positioned on an opposite side of the second balloon from the second marker. In a first configuration, the second balloon may be filled with barium sulfate. Each of the first port and the second port may be configured to couple to a syringe. The second balloon may be configured to transition between a fully retracted configuration and a fully expanded configuration. The second balloon may have a diameter between 7 millimeters and 12 millimeters in the fully expanded configuration. The medical device may further comprise a third balloon positioned proximate to the second balloon. The medical device may further comprise an opening fluidly connected to the lumen of the medical device. The opening may be configured to allow urine to pass therethrough.

In another aspect, a medical device for use during radiation therapy may comprise a longitudinal body configured to be positioned within a urethra. The longitudinal body may include a lumen, a distal end, and a proximal end. The first balloon may be coupled to a distal portion of the longitudinal body and fluidly connected to a first port at a proximal end of the longitudinal body. The first balloon may be configured to be positioned within a stomach. A second balloon may be positioned between the first balloon and a proximal end of the longitudinal body and fluidly connected to a second port at the proximal end of the longitudinal body. The second balloon may be configured to be positioned proximate to a penile bulb and is configured to block radiation.

Any of the aspects disclosed herein may include any of the following features, alone or in any combination. The medical device may be a urinary catheter. The medical device may further comprise at least one marking proximate to the second balloon. The at least one marking may be a first marking, and the device may further comprise a second marking. The first marking may be positioned on an opposite side of the second balloon from the second marking. In a first configuration, the second balloon may be filled with barium sulfate. An opening may be fluidly connected with the lumen of the medical device. The opening may be configured to allow urine to pass therethrough.

In an aspect, a method for performing radiation therapy on a patient may comprise positioning a medical device within a urethra of the patient, the medical device comprising: a longitudinal body configured to be positioned within a urethra. The longitudinal body may include a lumen, a distal end, and a proximal end. The medical device may further comprise a first balloon coupled to a distal portion of the longitudinal body and fluidly connected to a first port at a proximal end of the longitudinal body; and a second balloon positioned between the first balloon and a proximal end of the longitudinal body and fluidly connected to a second port at the proximal end of the longitudinal body. The method may further comprise positioning the second balloon proximate to a penile bulb of the patient; expanding the second balloon; and applying radiation to the patient.

Any of the aspects described herein may include any of the following features, alone or in any combination. The radiation therapy may be intensity modulated radiation therapy (IMRT) or stereotactic body radiation therapy (SBRT). The method may further comprise blocking the applied radiation using the second balloon. The method may further comprise inflating the second balloon with barium sulfate. The method may further comprise positioning at least one marker of the medical device proximate to the penile bulb. The method may further comprise inflating the first balloon within a stomach of the patient. The second balloon may be configured to transition between a fully retracted configuration and a fully expanded configuration. The second balloon may have a diameter between 7 millimeters and 12 millimeters in the fully expanded configuration.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a perspective view of an exemplary medical device, according to aspects of this disclosure.

FIG. 2 is a side, partial-cross-sectional view of the medical device of FIG. 1 positioned within a urethra, according to aspects of this disclosure.

FIG. 3 is a method of operation of the medical device of FIG. 1, according to aspects of this disclosure.

FIG. 4 is a perspective view of an exemplary medical device, according to aspects of this disclosure.

FIG. 5 is a perspective view of another exemplary medical device, according to aspects of this disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” “including,” or any other variation thereof, are intended to cover a non-exclusive inclusion, such that a process, method, article, or apparatus that comprises a list of elements does not include only those elements, but also may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure seek to improve a physician's ability to move a penile bulb of a patient and/or to block a penile bulb of a patient from receiving radiation using a medical device. The medical devices, systems, and methods discussed herein may help reduce exposure of the penile bulb, and/or other portions of a patient's anatomy, to radiation during radiation therapy, help reduce the likelihood of erective dysfunction developing in a patient after radiation therapy, and reduce damage to portions of a patient's anatomy during radiation therapy, among other aspects.

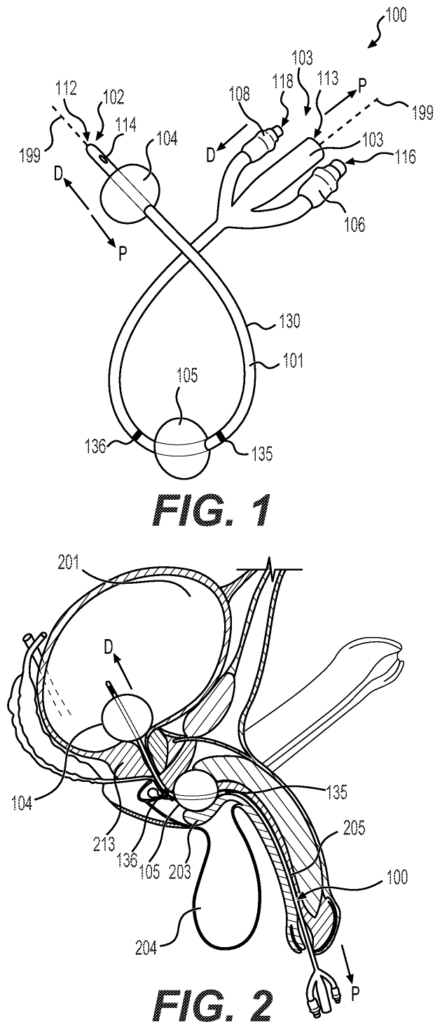

FIG. 1 shows a perspective view of a medical device 100. Medical device 100 may include a urinary catheter, such as a modified Foley catheter. Medical device 100 may include a longitudinal body 101 including a distal end 102 and a proximal end 103. Body 101 may be tubular with a one or more central lumens extending longitudinally through body 101 from a distal opening 112 (at distal end 102) to a proximal opening 113 (at proximal end 103). A central longitudinal axis 199 of medical device 100 may extend through opening 112, the central lumen, and opening 113. In some examples, one or more additional openings 114 may be positioned circumferentially around body 101 at distal end 102, and the one or more additional openings 114 may be fluidly connected to the central lumen. In some examples, the one or more additional openings 114, distal opening 112, and/or proximal opening 113 may be omitted. In such examples, medical device 100 may be removed immediately after a procedure. For example, medical device 100 may include the one or more additional openings 114 and proximal opening 113 but may omit distal opening 112.

A first balloon 104 may be positioned at a distal portion of body 101, proximal of distal end 102. First balloon 104 may be reversibly inflatable and may extend around a radially outer surface 130 of body 101, relative to central longitudinal axis 199 of body 101. First balloon 104 is shown in an expanded configuration in FIG. 1, and may be spherical when in an expanded configuration. In other examples, first balloon 104 may be an ellipsoid, tubular, rectangular, helical, or any other shape when in an expanded configuration. When first balloon 104 is in a retracted configuration, first balloon 104 may be substantially flush with radially outer surface 130 of body 101. First balloon 104 may have a unitary chamber, or a single chamber, within first balloon 104 configured to receive fluid. A first separate catheter (not shown) may fluidly connect first balloon 104 with a first valve/port 106 at proximal end 103 of body 101, and the first separate catheter may have a proximal opening 116. A first lumen of the first separate catheter may not be fluidly connected to the central lumen of body 101, and, in some examples, the first separate catheter may be positioned entirely within body 101. In some examples, central lumen of body 101 may include a plurality of catheters.

In some examples, first balloon 104 may have a limited capacity and only be configured to receive approximately 2.0 milliliters of fluid to approximately 3.0 milliliters of fluid. Additionally or alternatively, first balloon 104 may be configured to receive approximately 2.0 milliliters of fluid to approximately 100 milliliters of fluid, approximately 4.0 milliliters of fluid to approximately 80.0 milliliters of fluid, or approximately 5.0 to approximately 10.0 milliliters of fluid. However, such amounts are merely exemplary, and first balloon 104 may be configured to receive any suitable amount of fluid. The fluid received by first balloon 104 may include fluids such as sterile water or saline. When fluid is received in first balloon 104, it may be inflated to its fully inflated configuration (or fully expanded configuration). By using a syringe or similar instrument, a suitable volume of fluid may be injected through the first valve 106 and therethrough the first separate catheter into first balloon 104.

A second balloon 105 may be coupled to body 101 and positioned between distal end 102 of body 101 and proximal end 103 of body 101. Second balloon 105 may be reversibly inflatable and may extend around a radially outer surface 130 of body 101, relative to central longitudinal axis 199 of body 101. Second balloon 105 is shown in an expanded configuration in FIG. 1, and may be spherical when in an expanded configuration. In other examples, second balloon 105 may be an ellipsoid, tubular, rectangular, helical, or any other shape when in an expanded configuration. In some examples, second balloon 105 may have a diameter of approximately 7.0-12 millimeters when in a fully expanded configuration. When second balloon 105 is in a retracted configuration, second balloon 105 may be substantially flush with radially outer surface 130 of body 101. Second balloon 105 may have a unitary chamber within second balloon 105 configured to receive fluid. A second separate catheter (not shown) may fluidly connect second balloon 105 with a second valve/port 108 at proximal end 103 of body 101, and the second separate catheter may have a proximal opening 118. A second lumen of the second separate catheter may not be fluidly connected to the central lumen of body 101, and, in some examples, the second separate catheter may be positioned entirely within body 101. In some examples, the second separate catheter of body 101 may include a plurality of catheters.

In some examples, second balloon 105 may have a limited capacity and only be configured to receive between approximately 2.0 milliliters and approximately 10.0 milliliters of fluid (such as sterile water, saline, or contrast agent) to inflate second balloon 105 to its fully inflated configuration (or fully expanded configuration). The example capacity of second balloon 105 provided above is merely exemplary, and second balloon 105 may have alternative capacities. By using a syringe or similar instrument, a suitable volume of fluid may be injected through the second valve 108 and therethrough the second separate catheter into second balloon 105. Although second balloon 105 is shown at the approximate longitudinal midpoint of body 101, second balloon 105 may be positioned at any portion of body 101. In other examples, medical device 100 may include a plurality of second balloons 105 positioned between distal end 102 and proximal end 103. In some examples, medical device 100 may not include first balloon 104, and may only include second balloon 105 positioned between distal end 102 and proximal end 103.

Body 101 may include at least one marker 135, 136. As shown in FIG. 1, body 101 may include two markers 135, 136. Markers 135, 136 may be positioned on body 101 adjacent to second balloon 105. Markers 135, 136 may be visible by the human eye, under a computerized tomography (CT) scan, and/or under an x-ray scan. Markers 135, 136 may extend at least partially circumferentially around body 101, and, in some examples, may extend entirely around body 101. Markers 135, 136 may include tungsten or other heavy metals to block x-rays and allow a user to visualize markers 135, 136 under an x-ray scan, a CT scan, or other medical image. In some examples, markers 135, 136 may include metal rings extending around body 101. Markers 135, 136 may be positioned on opposite sides of second balloon 105. For example, marker 135 may be positioned on a first side of second balloon 105 (e.g., distally of second balloon 105), and marker 136 may be positioned on a second side of balloon 105, opposite to the first side (e.g., proximally of second balloon 105).

FIG. 2 illustrates medical device 100 positioned within a urethra 205 of a patient, with portions of the patient's anatomy shown in cross-section. As shown in FIG. 2, first balloon 104 may transition to an expanded configuration once the first balloon 104 is positioned within a stomach 201. Stomach 201 may be a bladder stomach. By expanding first balloon 104 in stomach 201, medical device 100 may be prevented from moving proximally through urethra 205 and thus prevented from falling out of the patient's urethra, effectively anchoring medical device 100 to the patient. Second balloon 105 may be positioned proximate to the patient's penile bulb 203, may be positioned proximal of prostate gland 213, and may be positioned distal to scrotum 204. As shown in FIG. 2, when second balloon 105 transitions to an expanded configuration, penile bulb 203 may be, in some examples, pushed proximally, away from prostate gland 213.

Urine and other bodily fluids may travel through one or more of the one or more additional openings 114 or distal opening 112, through the central lumen of body 101, and exit from proximal opening 113 to drain the patient's stomach 201 of fluids.

In some examples, second balloon 105 may be configured to act as a shield from radiation and block radiation from moving through second balloon 105. For example, second balloon 105 may be made of a material that blocks radiation. In some examples, second balloon 105 may be filled with a fluid (e.g., liquid) that blocks radiation, such as barium sulfate or another heavy metal solution (e.g., contrast). To block radiation, second balloon 105 may deflect, absorb, and/or otherwise prevent radiation from traveling through second balloon 105. If second balloon 105 is filled with a fluid that blocks radiation, second balloon 105 may be flushed with saline or another fluid after radiation is applied. Thus, if medical device 100 is to remain within a patient's body between radiation treatments (e.g., to serve urinary drainage functionality), residual radiation-blocking agent (e.g., contrast agent, barium sulfate, or another solution) may be eliminated.

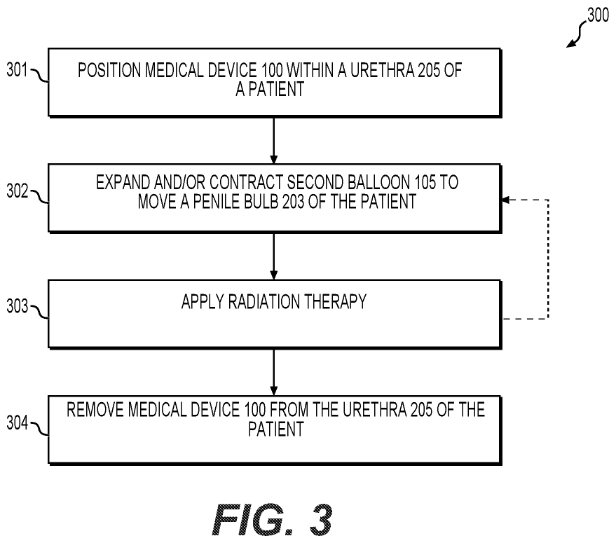

FIG. 3 illustrates a diagram of an exemplary method 300 of operating medical device 100. In step 301, a user may first position medical device 100 within a urethra 205 of a patient. For example, a user may insert distal end 102 of medical device 100 into an opening of urethra 205 and proceed to advance distal end 102 distally, toward stomach 201. In step 301, a user may visualize medical device 100, and, in at least some examples, markers 135, 136, under a CT scan or x-ray scan (or other type of imaging) of the patient. In some examples, the user may insert medical device 100 into urethra 205 and push medical device 100 distally through urethra 205 until markers 135, 136 are proximate to penile bulb 203. In some examples, one marker 135, 136 may be distal to penile bulb 203 and one marker 135, 136 may be proximal of penile bulb 203. Each of first balloon 104 and second balloon 105 may be in a retracted configuration during the insertion of medical device 100 into urethra 205 and positioning of second balloon 105 proximate to penile bulb 203. In some examples, prior to insertion of medical device 100 into patient, a user may determine a distance from stomach 201 to penile bulb 203 of the patient, and position second balloon 105 approximately the same distance from first balloon 104 as the distance from stomach 201 to penile bulb 203. In some examples, to anchor medical device 100 to the patient, the user may transition first balloon 104 to an expanded configuration within stomach 201 to prevent proximal movement of medical device 100 through urethra 205. For example, a user may fill first balloon 104 with fluid to transition first balloon 104 to an expanded configuration.

In step 302, the user may fill second balloon 105 with fluid to transition second balloon 105 from a retracted configuration to an expanded configuration. Since penile bulb 203 is proximate to second balloon 105, penile bulb 203 may move from the force applied by second balloon 105 as it expands. The user may expand and contract second balloon 105 to change the position of penile bulb 203. In some examples, the user may position second balloon 105 in between a source of radiation and penile bulb 203 (e.g., distal to penile bulb 203 or any other positioning) to block radiation applied by the source of radiation from hitting penile bulb 203. For example, the user may position second balloon 105 in between a source of radiation and penile bulb 203, and then the user may fill second balloon 105 with a fluid that blocks radiation. In other words, an imaginary line extending between the source of radiation and penile bulb may intersect balloon 105 after balloon 105 is positioned. In step 303, the user may then apply radiation therapy to the patient, for example by directing radiation at prostate gland 213. Then, in step 304, user may remove medical device 100 from urethra 205 of the patient.

In some examples, medical device 100 may remain in a patient after radiation therapy. For example, the patient may retain medical device 100 within urethra 205 during an entire course of treatment including multiple radiation therapy sessions. In some examples, medical device 100 may remain within a patient for a few days, a week, two weeks, a month, or any other time period. If medical device 100 is to remain in the patient's body, then, as discussed above, second balloon 105 may be flushed (e.g., with saline or another fluid) to remove residual fluid that blocks radiation and to allow urine to drain from the patient through medical device 100.

Various aspects of medical device 100, for example, body 101, first balloon 104, and second balloon 105, may have a low cost and may be disposable (i.e., a single use device).

Medical device 100 may be used in any type of radiation therapy, such as intensity modulated radiation therapy (IMRT), external beam radiation therapy (EBRT), proton beam therapy, image-guided radiation therapy (IGRT), brachytherapy, radium-223 therapy, and/or stereotactic body radiation therapy (SBRT) or stereotactic ablative radiation therapy (SABR).

In some aspects, medical device 100 may be positioned and repositioned before expanding first balloon 104 or second balloon 105. This positioning and repositioning may be done under indirect visualization, for example, via CT imaging.

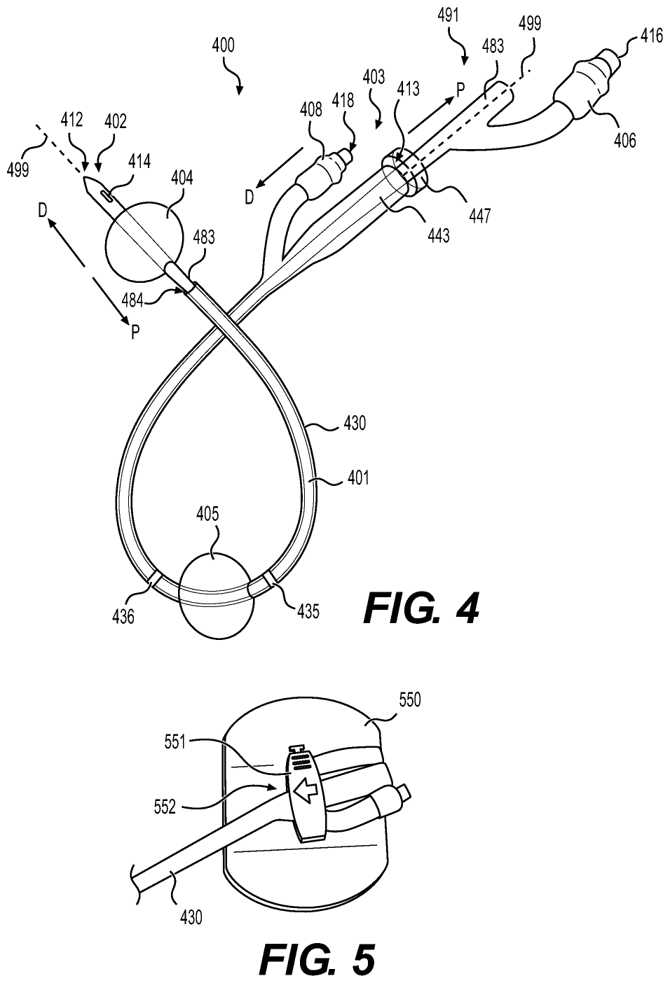

FIG. 4 shows a perspective view of another exemplary medical device 400, and medical device 400 may have any of the features and attributes described hereinabove with relation to medical device 100. Medical device 400 may include a urinary catheter, such as a modified Foley catheter. Medical device 400 may include a longitudinal body 401 including a distal end 402 and a proximal end 403. Body 401 may be tubular with a one or more central lumens extending longitudinally through body 401. A central longitudinal axis 499 of medical device 400 may extend through body 401. Longitudinal body 401 may include a first longitudinal body 483 and a second longitudinal body 430, and second longitudinal body 430 may be positioned around first longitudinal body 483. First longitudinal body 483 may include a distal opening 412 and a proximal opening 416, and second longitudinal body 430 may include a proximal opening 413 and a distal opening 484. Second longitudinal body 430 may be sized to receive first longitudinal body 483 within a central longitudinal lumen (extending from proximal opening 413 to distal opening 484) of second longitudinal body 430, and first longitudinal body 483 may be moveable proximally or distally within second longitudinal body 430.

In some examples, one or more additional openings 414 may be positioned circumferentially around body 483 at distal end 402, and the one or more additional openings 414 may be fluidly connected to a central lumen of second longitudinal body 483. In some examples, the one or more additional openings 414, distal opening 412, and/or proximal opening 413 may be omitted. In such examples, medical device 400 may be removed immediately after a procedure. For example, medical device 400 may include the one or more additional openings 414 and proximal opening 413 but may omit distal opening 412.

A first balloon 404 may be positioned at a distal portion of second longitudinal body 483, proximal of distal end 402. First balloon 404 may have any of the features of first balloon 104 described herein, and may be reversibly inflatable and may extend around a radially outer surface of body 483, relative to central longitudinal axis 499 of body 483. First balloon 404 is shown in an expanded configuration in FIG. 4, and may be spherical when in an expanded configuration. When first balloon 404 is in a retracted configuration, first balloon 404 may be substantially flush with radially outer surface of second longitudinal body 483, and first balloon 404 may be pushed through second longitudinal body 430 when in a retracted configuration (e.g. moved proximally or distally through a central lumen of second longitudinal body 430). First balloon 404 may have a unitary chamber, or a single chamber, within first balloon 404 configured to receive fluid. A first separate catheter (not shown) may fluidly connect first balloon 404 with a first valve/port 406 at proximal end of body 483, and the first separate catheter may have a proximal opening 416. A first lumen of the first separate catheter may not be fluidly connected to the central lumen of first longitudinal body 483, and, in some examples, the first separate catheter may be positioned entirely within first longitudinal body 483. In some examples, central lumen of first longitudinal body 483 may include a plurality of catheters.

When fluid is received in first balloon 404, it may be inflated to its fully inflated configuration (or fully expanded configuration). By using a syringe or similar instrument, a suitable volume of fluid may be injected through the first valve 406 and therethrough the first separate catheter into first balloon 404.

A second balloon 405 may be coupled to second longitudinal body 430 and positioned between distal end 402 of body 430 and proximal end 443 of body 430. Second balloon 405 may be reversibly inflatable and may extend around a radially outer surface of body 430, relative to central longitudinal axis 499. Second balloon 405 is shown in an expanded configuration in FIG. 4, and may be spherical when in an expanded configuration. Second balloon 405 may have any of the features of second balloon 105 described herein. When second balloon 405 is in a retracted configuration, second balloon 405 may be substantially flush with radially outer surface of body 430. Second balloon 405 may have a unitary chamber within second balloon 405 configured to receive fluid. A second separate catheter (not shown) may fluidly connect second balloon 405 with a second valve/port 408 at proximal end 443 of body 430, and the second separate catheter may have a proximal opening 418. A second lumen of the second separate catheter may not be fluidly connected to the central lumen of body 430, and, in some examples, the second separate catheter may be positioned entirely within body 430. In some examples, the second separate catheter of body 430 may include a plurality of catheters. In other examples, a second lumen may be positioned within a portion of second longitudinal body 430, such as within a wall that forms a central lumen of second longitudinal body 430, and this second lumen may be fluidically connected to second balloon 405 and second valve/port 408.

By using a syringe or similar instrument, a suitable volume of fluid may be injected through the second valve 408 and therethrough the second separate catheter into second balloon 405. Although second balloon 405 is shown at the approximate longitudinal midpoint of body 430, second balloon 405 may be positioned at any portion of body 430. In other examples, medical device 400 may include a plurality of second balloons 405 positioned around second longitudinal body 430.

A ring 447 may be coupled to second longitudinal body 430 at a proximal portion of second longitudinal body 430, and ring 447 may be flush with and/or create a seal around first longitudinal body 483. First longitudinal body 483 may be moveable distally and proximally through ring 447 while ring 447 maintains a seal between ring 447 and first longitudinal body 483. Markers 435, 436 may be positioned around second longitudinal body 430, and may have any of the features of markers 135, 136 described herein.

FIG. 5 illustrates a locking device 550 including a locking clasp 551. Locking device 550 may be configured to couple to and lock medical device 400, specifically by locking clasp 551 wrapping around second longitudinal body 430 (and first longitudinal body 483 positioned within second longitudinal body 430) and holding medical device 400. A lumen 552 of locking clasp 551 may be configured to receive first longitudinal body 483 and second longitudinal body 430. Locking device 550 may be configured to couple to skin of a patient, such as via an adhesive, strap or other coupling mechanism. Locking device 550 may allow a user to lock medical device 400 in a position, and may prevent movement of first longitudinal body 483 relative to second longitudinal body 430 when first longitudinal body 483 and second longitudinal body 430 are coupled to locking device 550 via locking clasp 551.

During operation, medical device 400 may allow a user to reposition first balloon 404 and/or second balloon 405, for example by moving first balloon 404 closer or farther away from second balloon 405 while device 400 is positioned within a body of a patient. Since first body 483 may be moved or slid through second longitudinal body 430, a user may move first balloon 404 proximally or distally relative to second balloon 405 by pushing or pulling a proximal portion 491 (which is exposed outside of second longitudinal body 430) of first longitudinal body 483. Once the user has positioned first balloon 404 at an appropriate position relative to second balloon 405 via the movement of first longitudinal body 483 relative to second longitudinal body 430, the user may lock medical device 400 at the position via locking device 550, and may then couple locking device 550 to the patient or another part of the operating room (e.g. a patient's bed, a surgical tool, a control unit, etc.).

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device for use during radiation therapy, comprising:
a longitudinal body configured to be positioned within a urethra, wherein the longitudinal body includes a lumen, a distal end, and a proximal end;
a first balloon coupled to a distal portion of the longitudinal body and fluidly connected to a first port at the proximal end of the longitudinal body; and
a second balloon positioned between the first balloon and the proximal end of the longitudinal body and fluidly connected to a second port at the proximal end of the longitudinal body.

2. The medical device of claim 1, wherein the medical device is a urinary catheter.

3. The medical device of claim 1, further comprising at least one marker proximate to the second balloon.

4. The medical device of claim 3, wherein the at least one marker is a first marker, and the medical device further comprises a second marker; wherein the first marker is positioned on an opposite side of the second balloon from the second marker.

5. The medical device of claim 1, wherein, in a first configuration, the second balloon is filled with barium sulfate.

6. The medical device of claim 1, wherein each of the first port and the second port is configured to couple to a syringe.

7. The medical device of claim 1, wherein the second balloon is configured to transition between a fully retracted configuration and a fully expanded configuration, wherein the second balloon has a diameter between 7 millimeters and 12 millimeters in the fully expanded configuration.

8. The medical device of claim 1, further comprising a third balloon positioned proximate to the second balloon.

9. A method for performing radiation therapy on a patient, the method comprising:
positioning a medical device within a urethra of the patient, the medical device comprising:
a longitudinal body configured to be positioned within a urethra, wherein the longitudinal body includes a lumen, a distal end, and a proximal end;
a first balloon coupled to a distal portion of the longitudinal body and fluidly connected to a first port at a proximal end of the longitudinal body; and
a second balloon positioned between the first balloon and a proximal end of the longitudinal body and fluidly connected to a second port at the proximal end of the longitudinal body;
positioning the second balloon proximate to a penile bulb of the patient;
expanding the second balloon; and
applying radiation to the patient.

10. The method of claim 9, wherein the radiation therapy is intensity modulated radiation therapy (IMRT) or stereotactic body radiation therapy (SBRT).

11. The method of claim 9, further comprising blocking the applied radiation using the second balloon.

12. The method of claim 9, further comprising inflating the second balloon with barium sulfate.

13. The method of claim 9, further comprising positioning at least one marker of the medical device proximate to the penile bulb.

14. The method of claim 9, further comprising inflating the first balloon within a stomach of the patient.

15. The method of claim 9, wherein the second balloon is configured to transition between a fully retracted configuration and a fully expanded configuration, wherein the second balloon has a diameter between 7 millimeters and 12 millimeters in the fully expanded configuration.

16. A medical device for use during radiation therapy, comprising:
a first longitudinal body configured to be positioned within a urethra, wherein the longitudinal body includes a first lumen, a first distal end, and a first proximal end;
a second longitudinal body configured to be positioned within the first longitudinal body, wherein the second longitudinal body includes a second lumen, a second distal end, and a second proximal end;
a first balloon coupled to a distal portion of the first longitudinal body and fluidly connected to a first port at the first proximal end of the first longitudinal body, wherein the first balloon is configured to be positioned with a stomach; and
a second balloon coupled to a distal portion of the second longitudinal body and fluidly connected to a second port at the second proximal end of the second longitudinal body, wherein the second balloon is configured to be positioned proximate to a penile bulb and is configured to block radiation.

17. The medical device of claim 16, wherein the medical device is a urinary catheter.

18. The medical device of claim 16, further comprising at least one marking proximate to the second balloon.

19. The medical device of claim 18, wherein the first longitudinal body configured to be moveable relative to the second longitudinal body.

20. The medical device of claim 16, wherein the second distal end of the second longitudinal body is proximal of the first balloon.
